(19) United States
(12) Patent Application Publication
(10) Pub. No.: US 2024/0252786 A1
(43) Pub. Date: Aug. 1, 2024

(54) APPARATUS AND METHODS FOR EVERTING CATHETERS WITH EXPANDABLE LUMENS

(71) Applicant: CrossBay Medical, Inc., San Diego, CA (US)

(72) Inventors: Steven R. BACICH, Half Moon Bay, CA (US); Piush VIDYARTHI, San Rafael, CA (US); Matthew Thomas YUREK, San Diego, CA (US)

(73) Assignee: CrossBay Medical, Inc., San Diego, CA (US)

(21) Appl. No.: 18/628,379

(22) Filed: Apr. 5, 2024

Related U.S. Application Data
(63) Continuation of application No. 17/214,496, filed on Mar. 26, 2021, now abandoned.
Application No. 17/214,496 is a continuation of application No. 16/796,589, filed on Feb. 20, 2020, now abandoned.
Application No. 16/796,589 is a continuation of application No. PCT/US2018/049234, filed on Aug. 31, 2018.
(60) Provisional application No. 62/553,057, filed on Aug. 31, 2017.

(51) Int. Cl. A61M 25/01

(52) U.S. Cl. CPC A61M 25/0119; A61M 25/0125; A61M 25/0138; A61M 25/0155; A61M 2210/14

(57) ABSTRACT

An everting catheter with an expandable inner lumen for the passage of instruments or other devices is described. The catheter can have an inflatable everting balloon. When inflated, the everting balloon can define a channel or passageway, via an inner balloon lumen, into a target site. Instruments can be delivered to the target site through the channel defined by the inflated everting balloon.

### CROSS-REFERENCE TO RELATED APPLICATION

This application is a continuation of U.S. patent application Ser. No. 17/214,496, filed Mar. 26, 2021, which is a continuation of U.S. patent application Ser. No. 16/796,589 filed Feb. 20, 2020 (now abandoned), which is a continuation of International Application No. PCT/US2018/049234, filed Aug. 31, 2018, which claims priority to U.S. Provisional Application No. 62/553,057, filed Aug. 31, 2017, which are incorporated by reference herein in their entireties.

### BACKGROUND

### 1. Technical Field

An everting catheter with an expandable inner lumen for the passage of instruments or other devices is described. The disclosed devices and methods have applications for the passage of endoscopes, IUD inserters, biopsy instruments, graspers, stent delivery systems, and other surgical instrumentation including electrocautery devices and tissue retraction and expansion devices. In these applications, the everting catheter obtains access to the bodily lumen or cavity with an inner lumen that has a first profile that is smaller than a second profile when a surgical instrument, device, endoscope, or IUD inserter is placed through it. The expandable inner lumen can be or have a conduit for the passage of the instrument, device, endoscope, or IUD inserter that can avoid or minimize the risk of perforation, simplify the procedure for the physician, and increase patient comfort. The inner lumen can expand when the objects are placed through the inner lumen.

The everting balloon can tear or peel along an intentional weakness, perforation, or split, for example, created by a mechanical implement. The tearing or peeling of the everting balloon can increase the inner lumen diameter and allow for a larger profile device to pass through the everting catheter.

The everting catheter can be used to place an echogenic tubal patency system within an everting catheter. The everting catheter can be used to place an aspiration type biopsy device within an everting catheter. The inner lumen of the everting catheter can have various integrated biopsy devices. The everting catheter can have combinations of the features and elements disclosed herein.

The everting catheter can be used for accessing and treating vessels, bodily cavities and lumens, for example, that are tortuous, narrow or stenotic, or where passing traditional guidewires, catheters, and probes is difficult. The everting catheter can be used in anatomical situations (e.g., the cervix and the uterine cavity) in which the risk of perforation is high. The everting catheter can be used where the passage of catheters and instruments carries the risk of transferring bacteria or infectious materials inside the body. The everting catheter can travel without friction in a biological lumen or cavity (e.g., the uterine cavity from the vagina and exocervix) and minimize or eliminate the risk of passing infectious materials within the body.

The everting catheter can be used in the cervix, fallopian tubes for contraception, the urethra and bladder for evaluation and treatment of urological disorders, the uterine cavity for the treatment of excessive menorrhagia, the arterial system for the treatment of plaque, the venous system for the treatment of valve disorders, sinus passageways for the treatment of sinusitis, and additional passageways in the mammalian body including the urethra, ureters, bile ducts, mammary ducts, spinal cord, gastrointestinal tract for the treatment of disorders or tissue therapy.

The everting catheter can be used for accessing and treating vessels and cavities in combination with other instruments, media, therapeutic agents, and devices which can be equally delivered or placed for treatment or therapy through the expandable inner lumen. Several of these combinations will be explained in the following descriptions.

### 2. Related Art

For physicians and medical professionals, accessing systems for vessels and bodily cavities in patients have typically used various guidewire and catheter technologies. In the techniques described above, the methods involved pushing an object, guidewire, probe, mandrel, or device itself through the vessel to gain access to a desired region in the body. The result of pushing an object, mandrel, or device creates shear forces on the lumen wall. In some cases the shear forces can result in trauma, pain for the patient, or perforation. In addition, the tortuosity and attributes of the physical anatomy may make access to the desired therapeutic site difficult and challenging.

In contrast, another access technology is referred to as an everting catheter. Everting catheters utilize a traversing action in which a balloon is inverted and with the influence of hydraulic pressure created by a compressible or incompressible fluid or media rolls inside out or everts with a propulsion force through the vessel. Everting balloons have been referred to as rolling or outrolling balloons, evaginating membranes, toposcopic catheters, or linear everting catheters. In practice everting balloons, due to their property of traversing vessels, cavities, tubes, or ducts in a frictionless manner, could provide physicians an ability to place instruments in a bodily cavity or lumen with a reduced risk of traumatic forces on the lumen or cavity wall. An everting balloon can traverse a tube without imparting any shear forces on the wall being traversed. Because of this action and lack of shear forces, resultant trauma can be reduced and the risk of perforation reduced. In addition, as a result of the mechanism of travel through a vessel, material and substances in the proximal portion of the tube or vessel are not pushed or advanced forward to a more distal portion of the tube or vessel. In addition, as the everting catheter deploys inside out, uncontaminated or untouched balloon material is placed inside the vessel wall. In the inverted or undeployed state, the balloon is often housed inside the catheter body and typically will not come into contact with the patient or physician. As the balloon is pressurized and everted, the balloon material rolls inside out, usually without contacting any element outside of the vessel. The method of access for an everting balloon can be more comfortable for the patient since the hydraulic forces “pull” the balloon membrane through the vessel or duct as opposed to a standard catheter that needs to be “pushed” into and through the vessel or duct.

Due to its ability to navigate tortuous anatomy and gain access to difficult regions of the body, the everting balloon can be a useful tool for physicians to provide therapeutic tools to these regions. One limitation with everting catheters is the size of the inner lumen for the passage of instruments, endoscope, or devices. If the inner lumen is large for the passage of the desired instrument, endoscope, or device, the overall access profile of the everting catheter becomes large enough to affect the performance of the everting system. The size profile of an instrument in terms of its outer diameter, and its flexibility, pushability, torqueability, and articulation are all physical attributes that contribute to how easily an instrument can be passed into the body. The size of an instrument that can be passed within an everting catheter is dictated by the internal diameter of its inner catheter and the maximum diameter of its everting balloon membrane. It may be desirable to maintain a low profile with the access system. As an example, making an everting balloon 7 mm in diameter might prove to be too large for consistent access within certain bodily lumens.

The inner lumen of the inner catheter can be a supporting structure for the passage of larger profile instruments. This function is not adequately addressed by the everting balloon membrane itself since an everting balloon membrane that is thin, flexible, and can easily evert to navigate tortuous anatomy. For everting catheters, the everting balloon pulls the inner catheter into the bodily cavity or lumen and the inner catheter can then become the guide structure for the passage of larger profile instruments.

### SUMMARY

A system for accessing a bodily cavity or lumen with an everting catheter and an everting balloon is disclosed. The system can have an inner catheter lumen that can access the bodily cavity or lumen in a first profile that is smaller than a second profile. When in the second profile, an instrument can be placed through the system, for example forcing the system into the second profile.

The system can have an inner catheter that can be pleated when in the first profile. The system can have an inner catheter that can be folded when in the first profile. The system can have an inner catheter that can be unexpanded in the first profile and can be expanded to a second profile. The instrument can be or have an endoscope. The instrument can be or have an IUD inserter.

The second profile can include a tearing of the everting balloon. The everting balloon can have an intentional weakness in the wall of the balloon membrane. The tearing of the everting balloon can include the use of a mechanical implement that is active when the instrument is placed through the inner catheter. For example, the mechanical implement can directly tear the everting balloon. The everting balloon can be weakened, for example by being thinned and/or with perforations, along the tear line.

Once access is achieved, the conduit created by the inner lumen can be exploited by the physician for the access of larger profile devices, instruments, or endoscopes. An inner lumen can then expand to allow for the access and passage of the instrument, device, or endoscope benefits the physician and patient.

The inner catheter can be delivered to the bodily cavity or lumen by the everting catheter in a smaller, more flexible profile. The inner catheter that contains the property of expansion with a second profile can accommodate larger profile instruments, endoscopes, or devices (e.g., ones that could not be inserted into the bodily cavity or lumen directly without undue trauma, difficulty, or higher risk to the patient).

The expansion of the inner lumen can rely on the intentional tearing or perforation of the everting balloon itself. The intentional tearing of the balloon membrane can allow a larger profile device to gain access. The intentional tearing can be done in a manner that does not leave remnants of the balloon membrane within the bodily cavity or lumen and may not tear the inner catheter lumen to maintain a consistent track or passage for the advancement and retraction of the desired instrument. The everting catheter system can be single use or multi use. For example, the tearing of the everting balloon membrane and the expansion of the inner catheter lumen may dictate that the everting catheter system is single use which may be helpful for patient safety and to the reduce risk of infection.

The everting balloon can be adapted to become the therapeutic tool or device once placed in the desired location in the body. For example, biopsy devices that are integrated with the everting catheter systems are described. For this application, the portion of the biopsy instrument that is used for sample collection may only be exposed to the bodily cavity or lumen when the everting system is in the proper location in the body by everting through the inner lumen. After tissue, fluid, cellular materials, or combinations of all of these elements of specimen collection are contained within the biopsy instrument of the everting catheter system, the balloon membrane can then be inverted to capture and contain the collected specimen to preserve the integrity of the specimen without contamination during the withdrawal of the everting system from the bodily cavity or lumen. Maintaining the collected specimen within the inverted catheter system can protect the integrity of specimen during transport to the examination area or diagnostic tissue fixing and staining location.

A system for accessing a body cavity or body lumen is disclosed. The system can have an everting catheter system. The everting catheter system can have an inner catheter, an outer catheter, and an everting balloon. The inner catheter can define an inner lumen that can accesses or be in fluid communication with the body cavity or body lumen. The inner catheter can have a radially compressed first profile (e.g., cross-sectional shape and size) and a radially expanded second profile. The first profile can be smaller than the second profile. The inner catheter can be folded or pleated when the inner catheter is in the first profile. The system can have an instrument placed through the inner lumen. When the instrument is in the inner lumen, the inner catheter can be radially expanded in in the second profile around the instrument.

The inner catheter can be radially unexpanded in the first profile and radially expanded in the second profile. The instrument can have or be an endoscope, a cytology brush, an IUD inserter, an aspiration-type biopsy device, a shaver, or combinations thereof.

When the inner catheter is in the second profile, the everting balloon can be torn at an intentional weakness in the wall of the everting balloon. The intentional weakness can have or be a perforation and/or a bond point, line or area. The tearing of the everting balloon can include the use of a mechanical implement (e.g., scissors, a knife edge, scalpel or other blade) that can be active when the instrument is placed through the inner balloon lumen.

A method for accessing a body cavity or lumen is disclosed. The method can include inserting an everting catheter system into the body. The everting catheter system can have an inner catheter defining an inner lumen, an outer catheter, and an everting balloon defining a balloon lumen. The inner catheter can be in the balloon lumen. The method can include deploying the everting balloon out of the distal end of the everting catheter system and into the body cavity or lumen. The deploying can include positioning the inner catheter in the body cavity or lumen in a first profile that is smaller than a second profile. The inner catheter can be pleated or folded when the inner catheter is in the first profile. The method can include inserting an instrument through the inner lumen. The inner catheter can have the second, radially expanded profile around the instrument when the instrument is inserted in the inner lumen.

The method can include tearing the everting balloon. The tearing can be concurrent with the instrument being positioned in the inner balloon lumen. The tearing can be caused by the radial expansion of the everting balloon due to the stretching of the everting balloon to radially accommodate the instrument. The tearing can occur during the inserting of the instrument into the inner lumen. The tearing can include tearing an intentional weakness in the everting balloon. The intentional weakness can have or be a perforation and/or a bond point, line or area.

### BRIEF DESCRIPTION OF THE DRAWINGS

FIG. 1 is a cross-sectional view of a variation of an expandable inner lumen within an everting catheter.

FIG. 2 is a cross-sectional view of a variation of a method for inserting an instrument into the inner catheter and expanding the inner lumen of the inner catheter.

FIG. 3 is a cross-sectional view of a variation of a method for inserting an instrument into and fully through the expandable inner lumen.

FIG. 4 is a cross-sectional view of a variation of an everting balloon surrounding an expandable portion of the inner catheter.

FIGS. 5a, 5b, and 5c are side, proximal axial (section A-A), and distal axial (section B-B) views, respectively, of a variation of an expandable pleated inner lumen and surrounding tubing (e.g., the inner catheter).

FIGS. 6a, 6b, and 6c are axial views of a variation of a method, in respective sequential temporal order, of a method for manufacturing (i.e., the construction) of an expandable inner lumen tubing with folds.

FIG. 7 illustrates a variation of an expandable inner lumen with braided tubing.

FIG. 8a illustrates a variation of an everting catheter system with an integrated biopsy brush.

FIG. 8b illustrates the everting catheter system with an integrated biopsy brush of FIG. 8a with the inner catheter in a slightly advanced configuration.

FIG. 8c is a close-up view of a variation of an everted balloon extending from the distal end of the outer catheter.

FIG. 8d illustrates a variation of an everting catheter with an integrated biopsy brush slightly protruding from the everted balloon.

FIG. 8e is a close-up view of the biopsy brush protruding beyond the everting balloon of FIG. 8d.

FIG. 8f illustrates the everting catheter of FIG. 8d with the integrated biopsy brush in a fully everted position.

FIG. 8g is a close-up view of the biopsy brush extended beyond the distal end of the everting balloon of FIG. 8f.

FIG. 8h is a close-up view of the knob of the cytology brush and the proximal end of the inner catheter of FIG. 8f.

FIG. 8i is a close-up view of the cytology brush of FIG. 8f at the beginning of the inversion of the everting balloon.

FIG. 8j is a close-up view of the cytology brush of FIG. 8f fully enveloped during inversion of the everting balloon.

FIG. 9a is a cross-sectional view of a variation of a curette within the distal end of the everting balloon and on target tissue.

FIG. 9b is a cross-sectional view of a variation of the curette enveloped by the everting balloon upon inversion.

FIG. 10 is a side view of a variation of a shaver within an everting balloon in the fully everted state, the everting balloon shown in cross-section.

FIGS. 11a through 11d illustrate a variation of a method of using the everting catheter system through a cervix.

FIGS. 12a through 12c illustrate a variation of a method of using the everting catheter system through a cervix.

### DETAILED DESCRIPTION

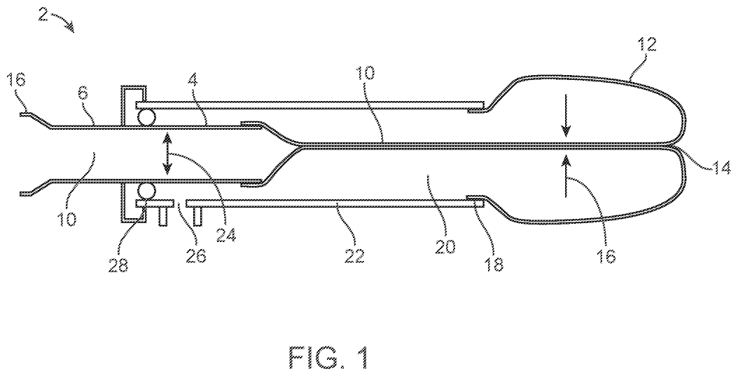

FIG. 1 illustrates that an everting catheter system 2 can be in an everted state with the balloon membrane connected to the outer catheter 22 at one end and attached to the inner catheter 6 at the other end. The balloon membrane or everting balloon 12 can be everted from the distal end of the outer catheter 22 upon the influence of hydraulic energy supplied by pressurized fluid media (not shown in FIG. 1). Once pressurized, the inner catheter 6 can advance or translate within the outer catheter 22. The distal end of the outer catheter 22 can have or be fitted with an acorn tip 56, for example, to limit the length of insertion into the bodily lumen, or an angled tip and/or an articulating tip, for example, to guide the introduction of the distal end opening into and through a bodily orifice, or to directionally place the distal end opening within a specific bodily orifice.

The everting balloon 12 can be inflated with a fluid pressure in an operating range from about 2 to about 5 atmospheres, or from about 0.25 to 1.9 atmospheres, or more than 5 atmospheres, for example, depending upon the application, the bodily lumen, or cavity being traversed, and degree of stenosis within the bodily lumen or cavity. The volume between the inner and outer catheters 22 can be an inflation reservoir 20. The inflation reservoir 20 can be pressurized at a fluid port 26 that can be connected to the annulus of the everting catheter system 2. The catheter system can have tubing connected to the fluid port 26, a stop cock 64, a fluid pressure source (e.g., a pump), and combinations thereof. The pressure source can be a syringe, an inflation device, a bellows, another supply of fluid, air, gas, or combination of this media, or combinations thereof. The pressure source can manually or automatically deliver fluid through or retract fluid from the fluid port 26 and provide it to the annulus of the inflation reservoir 20 inside the outer catheter 22. The everting catheter system 2 can have a scaling assembly 28, for example having a valve fitting with a gasket or o-ring seal inside the proximal end 42 of the outer catheter 22. The gasket or o-ring seal can allow for the translation of the inner catheter 6 during the pressurization state without leaking the pressurized fluid in the inflation reservoir 20.

The proximal terminal end of the inner catheter 6 can have an inner catheter proximal hub 8. Instruments 30 (e.g., devices, endoscopes, delivery channels, hysteroscopes) can be inserted into and delivered through the proximal hub 32.

All or part of the proximal portion of the inner catheter 6 can be non-expandable. This inner catheter non-expandable portion 4 can translate through the valve fitting and gasket or o-ring seal. As an example, the internal diameter 24 of the non-expandable portion of the inner catheter 6 can be about 3.2 mm with a wall thickness of about 0.1 mm.

An inner catheter expandable portion 40 can be attached to the distal end of the non-expandable portion of the inner catheter 6. The proximal end 42 of the inner catheter expandable portion 40 can have an internal diameter 16 of about 3.2 mm where it is attached to the non-expandable portion. The outer diameter of the inner length of the inner catheter expandable portion 40 can taper down to about 1.0 mm along the remainder of its length and at its distal end 46. The expandable portion of the inner catheter 6 can have a lower profile than the non-expandable portion of the inner catheter 6. The expandable portion of the inner catheter 6 can evert and access tight and tortuous anatomical locations in the body. The expandable portion of the inner catheter 6 can be an everting balloon 12.

The entire inner catheter 6 can be made from an expandable material. The valve fitting and seal on the outer catheter 22 can accommodate the size or profile of an instrument 30 (e.g., an endoscope) passed through the inner lumen of the inner catheter 6. The inner lumen 10 can be expandable. The inner lumen 10 can be defined by the radially inner-most surface of the inner catheter 6.

The expandable portion of the inner catheter 6 can be only that portion of the inner catheter 6 that exits the distal end of the outer catheter 22 (e.g., within the everting balloon 12). The everting balloon 12 can evert out of the distal end of the outer catheter 22. The everting balloon 12 can be inflated to a larger outer diameter than the outer diameter of the outer catheter 22. The proximal end 42 of the radially outer portion of the everting balloon 12 can be fixedly attached to the distal terminal end of the outer catheter 22 and an outer catheter-balloon connection 18, for example an epoxy, a length of a pinch-fit, a weld, or combinations thereof. The catheter-balloon connection can be a length along the circumferential, radially outer and/or radially inner, perimeter of the distal terminal end of the outer catheter 22. When in a deployed configuration, the distal terminal end of the expandable inner lumen 34 can be distal to the distal terminal end of the outer catheter 22.

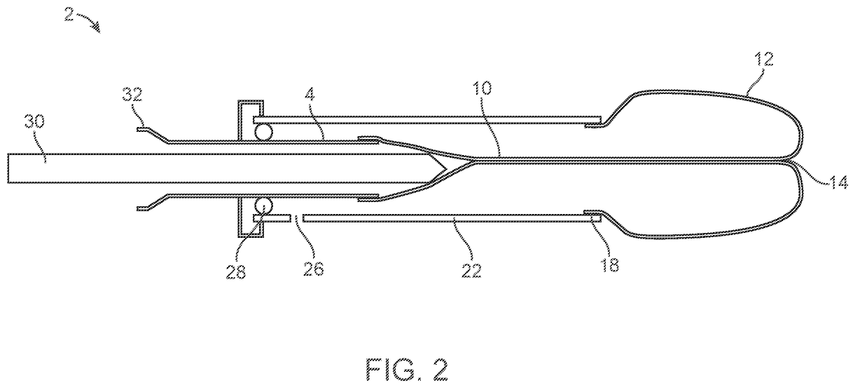

FIG. 2 illustrates that after the everting system can access a target site (e.g., desired location) in the body, for example by everting 116 the everting balloon 12, the instrument 30 can be inserted into and through the proximal hub 32. As the instrument 30 passes through the non-expandable portion of the inner catheter 6 and intubates the expandable portion of the inner catheter 6, the instrument 30 can resiliently open up the expandable portion of the inner catheter 6 to accommodate the size profile of the instrument 30. The inner catheter expandable portion 40 can be a conduit for the instrument passage. The inner catheter expandable portion 40 can be made from PET, polypropylene, nylon, HDPE (e.g., Teflon), other materials resistant to breakage from the instrument 30 and that have the lubricity to allow the instrument 30 to pass, and combinations thereof.

The expandable portion of the inner catheter 6 can be made from tubing made into a narrower profile by pleating, folding, made from a resilient elastomeric material, for example, that can be reinforced as described below, or combinations thereof. The internal lumen surface can have a lubricious coating and/or layer that can reduce that amount of friction between the instrument 30 and the inner surface of the inner lumen 10. The medical surgical instrument 30 can be another catheter, a device, an IUD inserter, an endoscope, a hysteroscope, or combinations thereof.

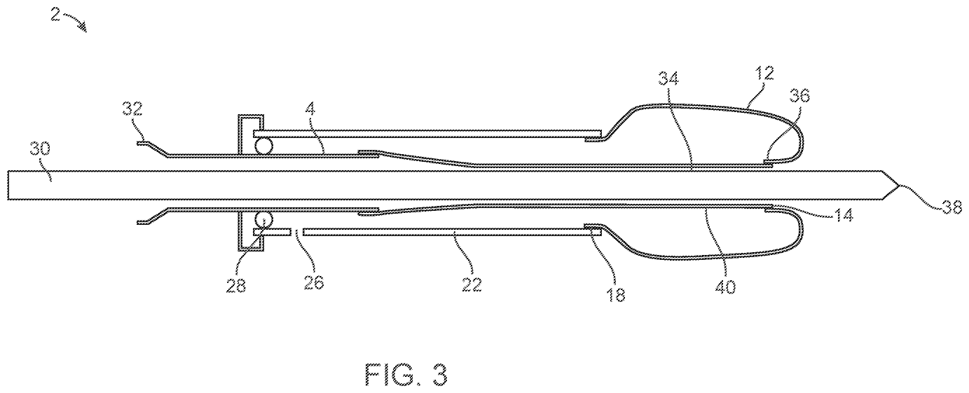

FIG. 3 illustrates that the instrument distal end 38 can be passed through the entire length of the everting catheter system 2. The instrument 30 can radially expand the entire length of the expandable inner lumen 34 and the everting balloon 12. The instrument 30 can exit or extend from an opening at the distal end of the expandable inner lumen 14 of the inner catheter expandable portion 40. The expandable inner lumen 34 can define a passageway for the insertion of the instrument 30 to the target site. The insertion of the instrument 30 can be done with the everting balloon 12 still pressurized or with the internal pressure reduced before, during, or after the insertion of the instrument 30 into the inner catheter 6. The internal pressure in the inflation reservoir 20 can be reduced to zero or negative pressure by applying an aspiration or vacuum source (e.g., drawing negative pressure on a syringe) to the fluid port 26 and internal annulus of the outer catheter 22.

The everting balloon 12 can be separate from the inner catheter 6. The everting balloon 12 can be attached to the distal end of the expandable inner lumen 14 by an elastomeric adhesive, thermally welding the balloon material to the expandable inner lumen material, or combinations thereof. The bond at the bond site 36 of the everting balloon 12 bonded to the distal end of the expandable inner lumen 14 can be flexible enough to allow the instrument 30 to pass through the bond site 36 area. The bond site 36 and/or everting balloon 12 can tear with the passage of the instrument 30, for example along an intentionally weakened line or area (e.g., the bond site, a perforated line or lines, or combinations thereof).

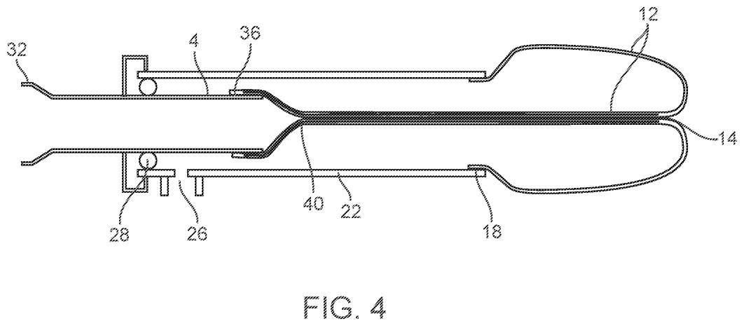

FIG. 4 illustrates that the expandable inner catheter 6 can be constructed without a distal end bond site 36 with the everting balloon 12. For example, the everting balloon 12 can extend radially outside of the length of the inner catheter expandable portion 40 and attach at a bond site to the inner catheter non-expandable portion 4, or the expandable inner catheter 6 and the everting balloon 12 can be made from a singular piece of the same material.

The everting balloon 12 and/or inner catheter expandable portion 40 can be bonded at a bond site 36 to the distal end of the non-expandable portion of the inner catheter 6. The balloon material that is immediately adjacent or surrounding the expandable portion of the inner catheter 6 can be left in its normal profile and not bonded at any location along the expandable portion of the inner catheter 6. As the inflation reservoir 20 or annulus is pressurized with fluid, the everting balloon 12 can tightly adhere and hold the inner catheter expandable portion 40 for the eversion process. When the expandable portion of the inner catheter 6 is expanded due to the insertion of an instrument 30 or endoscope through the inner lumen 10, the everting balloon 12 can expand to accommodate the radially expanding inner catheter expandable portion 40. The everting balloon 12 can be bonded to the inner catheter non-expandable portion 4 and not the inner catheter expandable portion 40, or to the inner catheter expandable portion 40 and not the inner catheter non-expandable portion 4, or to the inner catheter non-expandable portion 4 and the inner catheter expandable portion 40. For example, the everting balloon 12 can be unbonded to the any uneven surfaces of the distal end of the expandable portion of the inner catheter 6.

Continuing with the procedure using an everting catheter with an expandable inner lumen 34, once the instrument 30 is inserted through the entire length of the expandable portion of the inner lumen 10 and the everted balloon, the procedure can continue. As an example, if the instrument 30 is an endoscope, an irrigation source connected to the endoscope can supply fluid or gas distension media for improved visualization if necessary. A Touhy-Borst type adaptor connected onto the proximal hub 32 with the endoscope threaded through the internal seal can supply an additional source of irrigation media. The expandable inner lumen 34 can be used for multiple passages of instruments 30, or combinations thereof, depending upon the indication. The expansion diameter of the expandable inner lumen 34 during use can be substantially equal to the outer diameter of the instrument 30. As an example, if a 3.0 mm endoscope is inserted through the expandable inner lumen 34, the expandable inner lumen 34 diameter can be about 3.0 mm or a size to accommodate the outer diameter of the endoscope. Within the same procedure if a secondary instrument 30, for example having a diameter of about 3.2 mm, is inserted into the bodily cavity or lumen, the expandable inner lumen 34 can equally enlarge to accommodate the secondary instrument 30 outer diameter. Probes or dilators to increase the size of the bodily canal to reach the target anatomical site can be used or may not be used. For the physician, this allows for flexibility in selecting the appropriate instrument 30 size that is dependent upon the clinical need of the patient.

Once the instrument 30 or endoscopic procedure is completed, the instrument 30 or endoscope can be withdrawn separately, or together with the everting catheter system 2. The pressurization media in the internal annulus or reservoir chamber can be removed by negative pressure to reduce the profile of the everting balloon 12. Once the everting balloon 12 has retracted or is overwise deflated, the entire everting catheter system 2 and instrument 30 can be removed together from the patient's body. The instrument 30 can be inverted as the withdrawal mechanism.

A fluid can pressurize the internal annulus or inflation reservoir 20 of the everting catheter system 2. The distal end of the outer catheter 22 can be placed at the orifice of the target tissue 90. The everting balloon 12 can be everted to the target tissue 90 with the inner lumen 10 at a first (e.g., collapsed) profile. The instrument 30 can be inserted into the proximal hub 32 of the inner catheter 6 and advance or translate through and beyond the inner catheter expandable portion 40. The inner lumen 10 and inner catheter expandable portion 40 can assume a second (radially expanded) profile. The instrument 30 can then be used at the target site according to the desired procedure. The everting catheter system 2 and/or instrument 30 can then be withdrawn from the body.

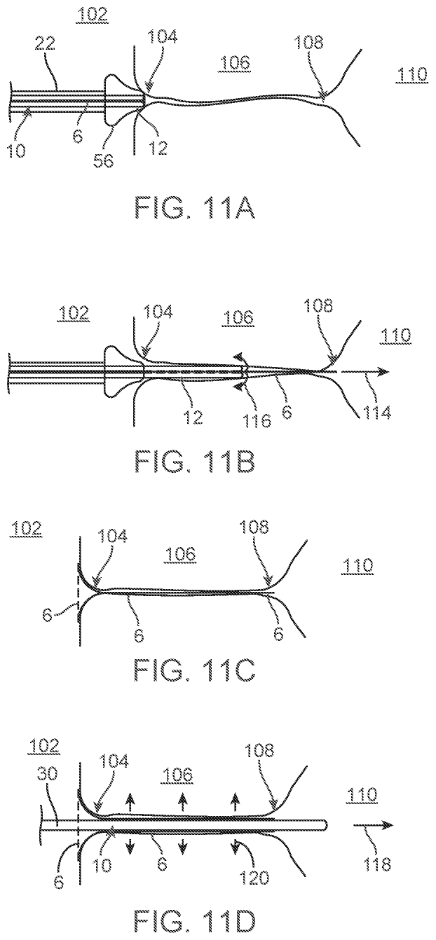

FIG. 11a illustrates that the distal end of the everting catheter system 2 in an uneverted configuration can be positioned—for example, while at least partially in a vagina 102—adjacent to the external os 104 of a cervix 106. The distal terminal end of the system can have an acorn tip 56. The distal terminal end of the inner catheter 6 can be positioned at the distal terminal end of the everting catheter system 2.

FIG. 11b illustrates that the inner catheter 6 can be translated, as shown by arrow 114, into the cervical canal, so that the distal end of the inner catheter 6 extends to or through the internal os 108 and possibly into the uterine cavity 110. During deployment of the inner catheter 6, the everting balloon 12 can evert. The inner catheter 6 can be ejected from the remainder of the everting catheter system 2 at two times the rate of the extension of the everting balloon 12. For example, the everting balloon 12 can be half-way though the cervix 106 when the inner catheter 6 is completely through the cervix 106. The distal half of the inner catheter 6 can be ejected from the everting balloon 12 lumen before deployment from and removal of the everting balloon 12 from the deployment site is complete.

FIG. 11c illustrates that the remainder of the everting catheter system 2 can be removed from the vagina 102. The inner catheter 6 can remain, in a radially compressed state, in and extending from the cervix 106. The proximal end 42 of the inner catheter 6 can be configured to expand into a radially expanded configuration, for example, exposing the entrance to the inner lumen 10 from proximal end 42 of the inner catheter 6.

FIG. 11d illustrates that the instrument 30 can be translated, as shown by arrow 118, through the inner lumen 10 of the inner catheter 6 while passing through the cervix 106. The cervical canal and the inner catheter 6 can radially expand, as shown by arrows, when the instrument 30 is translated through the cervical canal and the inner catheter 6. The inner catheter 6 can have folds 48, reinforcements, other features disclosed herein, or combinations thereof.

The instrument 30 can perform a procedure in and/or past the uterine cavity 110, such as delivering an IUD or other contraceptive device, reproductive material (e.g., sperm or a fertilized egg), performing a biopsy or visualization (e.g., with a hysteroscope), retrieving an egg, or combinations thereof. When complete, the instrument 30 can be removed from the cervix 106. A second instrument 30 can be translated through the inner lumen 10 of the inner catheter 6 after the first instrument 30 has been removed, or concurrent with the first instrument 30 being positioned in the inner lumen 10.

After the instruments 30 have been removed, the inner catheter 6 can be pulled out of the cervix 106.

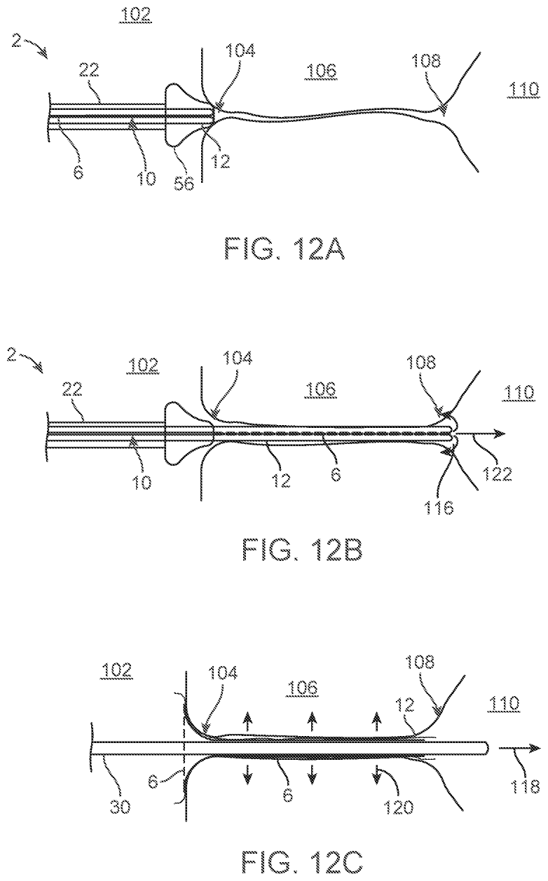

FIG. 12a illustrates that the inner catheter 6 can be loaded into the everting catheter system 2 before deployment so the distal end of the inner catheter 6 can be spaced proximally, for example by about half of the expected deployment length, from the distal terminal end of the everting catheter system 2 in an uneverted configuration.

FIG. 12b illustrates that before detachment from the remainder of the everting catheter system 2, the entire length of the inner catheter 6 can remain radially inside of the everting balloon 12, as the inner catheter 6 and balloon translate, as shown by arrow 122. The distal translation distance of the inner catheter 6 can be equal to or less than the balloon translation distance.

FIG. 12c illustrates that the everting balloon 12 can remain between the inner catheter 6 and the cervical wall when the instrument 30 is translated through the cervix 106. The everting balloon 12 can tear, for example along one or more intentionally weakened lines or areas, and can provide little or no radial force to resist the radial expansion 120 of the inner catheter 6 or cervix 106 when the instrument 30 is inserted through the inner catheter 6 (and everting balloon 12).

The everting balloon 12 can be removed with the inner catheter 6 when the inner catheter 6 is removed.

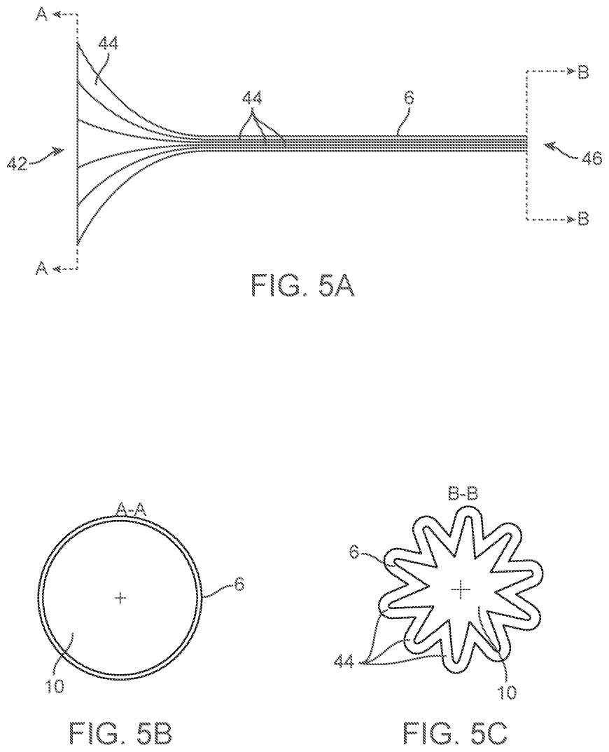

FIGS. 5a through 5c illustrate that the inner catheter expandable portion 40 and/or wall of the inner lumen 10 (e.g., tissue shield or protector) can have or be a tubing material. The tubing can have pleats 44 within the tubing material. The tubing material can be extruded with an extrusion die that provides the tubing in a pleat form. This pleat form can serve as the first profile (radially contracted) state, as shown in FIG. 5c, in the expandable inner lumen 34 application. The tubing material can then be radially enlarged into the second profile (radially expanded) state, as shown in FIG. 5b, when an instrument 30 (not shown in FIG. 5b) is placed within the internal lumen of the pleated tubing. The pleated form provided within the tubing wall opens and expands to a second profile or diameter as the instrument 30 or endoscope is passed through its internal lumen. The pleats 44 within the tubing can also be manufactured as a secondary operation within the tubing material as part of a molding, forming, or folding operation. The secondary operation can be done in a heated mold or as part of a drawing operation through a heated die that creates the pleated format within the tubing wall.

Pleated tubing made with larger pleats 44 in width can provide a greater change in the diameter for the second profile state. The width of the pleats 44 can be defined as a radial outward dimension from the central axis of the inner lumen 10. The number of pleats 44 themselves can influence the degree of change for a second profile state. Tubing made with one pleat can expand to a large second profile as that pleat can open to a second circumferential dimension upon the insertion of an instrument 30. Tubing made with more than one pleat or multiple pleats 44 can provide a great degree of change or may be easier to enlarge to a second profile state when an instrument 30 is passed through the internal lumen of the expandable inner catheter 6.

The pleats 44 can extend longitudinally along the inner catheter 6, parallel with the longitudinal axis. The pleats 44 can extend in a helix around the inner catheter 6. The pleats 44 can extend all or a part of a length of the inner catheter 6. The inner catheter 6 can be the devices taught in U.S. Pat. No. 5,772,628, which is incorporated by reference herein in its entirety. The inner catheter 6 can have a

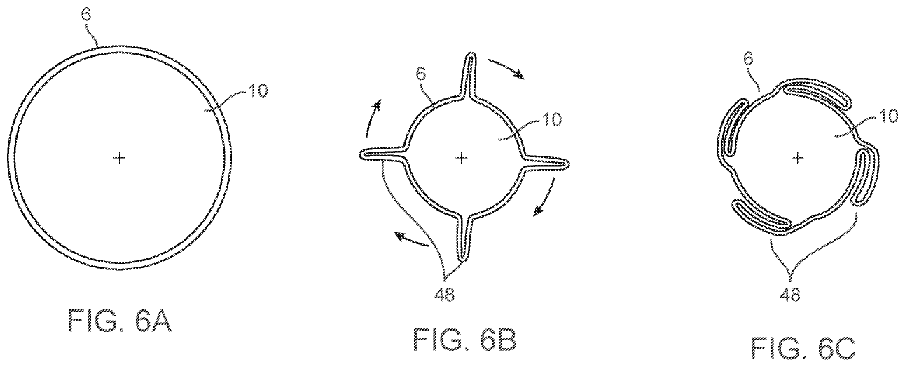

FIGS. 6a through 6 c illustrate that the expandable inner lumen 34 wall (e.g., the inner catheter 6) can have tubing material that is folded or has folds 48 placed within the tubing. FIG. 6a illustrates an axial view of the inner catheter 6 with folds 48 or pleats 44 in a radially expanded state. The inner catheter 6 can have an expandable foldable inner lumen 10. FIG. 6b illustrates that the tubing material can be compressed to a lower profile and excess material that makes up the circumference of the tubing can be folded into folds 48. FIG. 6c illustrates that once the material is folded, the folds 48 can be pressed to the outer periphery or circumference of the tubing, for example, to reduce the diameter of the tubing and provide the first profile state of the tubing. As an instrument 30 is passed through the expandable inner lumen 34 tubing made with folds 48, the folds 48 can unfold as the inner catheter 6 radially expands to the accommodate the outer diameter of the instrument 30.

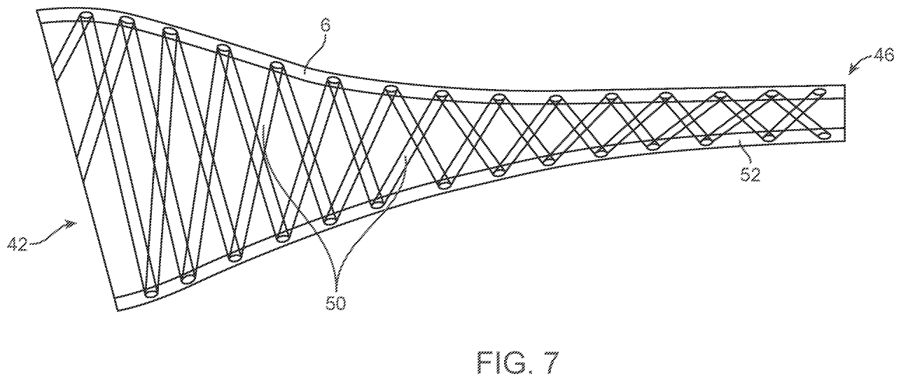

FIG. 7 illustrates that the expandable portion of the inner catheter 6 can be made with an elastomeric base 52, such as a solid, non-porous sheet, with a reinforcing braid. For example, the wall of the inner lumen 10 can have an expandable braid. The elastomeric material can be silicone, polyurethane, other elastomers, or combinations thereof. The elastomeric material can be resilient and return naturally to its first, low profile state after the instrument 30 or endoscope has been passed through its internal lumen, or non-resilient and configured in a low profile first state and undergoes some permanent deformation as an instrument 30 is passed through the inner lumen 10. The braid reinforcement 50 can reduce the potential for material tearing during instrument 30 passage. The braid can be made from nylon, Kevlar, polyester, nitinol, stainless steel, other polymer fiber, or combinations thereof. The internal lumen surface can be have a lubricious coating or layer that can reduce that amount of friction within the system for improved instrument 30 or endoscope passage. The inner catheter 6 can be radially compressed when in an unbiased state. The inner catheter 6 can be radially expanded at the proximal end 42 and radially compressed at the distal end 46, and can radially taper between the two ends when in an unbiased state. The inner catheter 6 or other wall of the inner lumen 10 can be made from an expandable elastomer. The elastomer can be fabricated without a reinforced braid with a uni-body construction that can, for example, relies on the elastic properties of the tubing material. The uni-body construction can be single construction tubing. An elastomeric tubing can have an application of a lubricious coating within the internal surface of the tubing, for example, to facilitate instrument 30 passage.

An elastomeric inner catheter 6 can be constructed of axially aligned reinforcements (co-linear with the central longitudinal axis of the tubing lumen), for example, that can protect the inner lumen 10 from tearing open during instrument 30 passage. The inner catheter 6 can have an elastomer. The longitudinally axially aligned reinforcements can be constructed from round or flat wire stainless steel, nitinol, polyester fibers, nylon, delrin mandrels, other polymer materials, or combinations thereof. The axially aligned reinforcements also serve to facilitate the passage of the instrument 30 or endoscope during insertion acting as rails for the instrument movement. The elastomeric inner lumen 10 expands circumferentially between the rails or axially aligned reinforcements.

The everting balloon 12 can have intentional weaknesses (e.g., perforations). As described above, the profile of the inserted instrument 30 or endoscope may not only force the expandable inner lumen 34 to reach a second larger profile, but the profile may exceed the normal operating diameter of the everting balloon 12 membrane. In these situations once access to the target anatomical site has been reached by the physician, and during instrument or endoscope insertion, the everting balloon 12 membrane may need to be torn to provide room or clearance for the instrument or endoscope to reach the bodily cavity or lumen. To facilitate the tearing of the everting balloon 12 membrane, intentional weaknesses in the balloon material itself may be placed to localize and direct the site of tear initiation. The tear can also be configured to propagate in a linear fashion co-linear with the central axis of the balloon material with no remnants or secondary breakage of balloon material that could be left in the body. To create these intentional weaknesses, a seam, indentation, crease, or small tear in the surface of the everting balloon 12 material can be made. Alternatively small pin holes can be made in the balloon material that does not allow fluid media to escape, but create an intentional weakness in the balloon wall thickness during high strain events. This can achieved with a minimum of one pin hole or multiple pin holes that form an intentional weakness in the balloon material to promote a predictable tear along a pre-determined path and location within the system. The tear is initiated by the increase in strain in the balloon material as a result of the increased diameter caused by the insertion of the instrument and endoscope. The pin holes can be made with a laser or mechanically driven by a fine implement. The pin holes can be made with a laser or mechanically driven by a fine implement. The laser holes or mechanically driven holes can be performed when the balloon material is placed into a pre-strained, stretched, or over-stretched condition to promote weakness in the material at the conditions of high strain.

Cross-linking the balloon material in an axial alignment can facilitate a balloon material tear along the central axis of the balloon.

The distal end bond site 36 at the outer catheter 22 to everting balloon 12 can be configured with a slit or split in the outer catheter 22 tubing to initiate a tear in the balloon material at the bond site 36. The same configuration of a split at the distal end of the inner catheter 6 to balloon bond site 36 can be configured.

A mechanical implement can be placed on the exterior surface of the expandable inner lumen 34 that reacts to the everting balloon 12 surface to initiate a tear in the balloon. This mechanical implement is active during the insertion of an instrument or endoscope.

The mechanical implement can be placed on the exterior surface of the distal end of the outer catheter 22 that reacts upon the increase diameter of the instrument or endoscope that serves to break or tear the balloon material. The mechanical implement can be a sharp surface made from metal or plastic that is made active or when it is pushed onto the balloon material by the insertion of the instrument or endoscope.

U.S. Pat. No. 9,028,401 is incorporated herein by reference in its entirety and describes an everting catheter with a dilating balloon. The intentional weakness in the everting balloon 12 can be pin holes or perforations for initiating a tear when inflating the dilating balloon. Small pin holes can be made in the balloon material that does not allow fluid media to escape during the eversion process, but create an intentional weakness in the balloon wall thickness during high strain events such that when the dilating balloon is inflated. This intentional weakness can achieved with a minimum of one pin hole or multiple pin holes that form an intentional weakness in the balloon material to promote a predictable tear along a pre-determined path and location within the system. The tear can be initiated by the increase in strain in the balloon material as a result of the increased diameter caused by the dilatation balloon. The pin holes can be made with a laser or mechanically driven by a fine implement. The laser holes or mechanically driven holes can be performed when the balloon material is placed into a pre-strained, stretched, or over-stretched condition to promote weakness in the material at the conditions of high strain.

The everting catheter system 2 can have an echogenic tubal patency system within the inner catheter 6. Echogenic tubal patency systems are described in US Patent Application Publication No. 2015/0133779, and U.S. Patent Application Nos. 62/005,355, and 62/302,194, which are all incorporated by reference herein in their entireties. The everting catheter with an echogenic tubal patency system can have an educator tube within the inner catheter of an everting catheter system 2. The distal end of the educator tube can be placed near the distal end of the inner catheter 6 when the everting catheter is nearly, or fully in, the everted state.

The distal end of the inner catheter 6 can have a venturi tube and throat.

The everting catheter system 2 can have a handle placed at the most proximal portion of the inner catheter 6. The handle can have an everting balloon 12 inflation port, an air infusion port, and a saline injection port for the fluid delivery of echogenic bubbles within the uterine cavity 110 for the assessment of tubal patency. The inner catheter 6 within the everting catheter system 2 can be or have a dual lumen tube with an opening near the distal portion of the inner catheter 6 with the opening directed towards the annulus of the outer catheter 22. The inflation port on the handle can deliver the pressurization media to the everting catheter system 2.

The everting catheter system 2 can have a biopsy device, for example a cytology brush 68, integrated within the everting catheter system 2. The biopsy device can be self-contained without the need of inserting or placing an accessory device within the everting catheter system 2. The everting catheter system 2 can provide greater access for the biopsy instrument by pulling the biopsy device into the target region of the anatomy. The everting catheter can deliver the biopsy device in a frictionless manner that can reduce or mitigate the amount of cellular material that can be pushed or scraped into the target tissue 90 region. The everting balloon 12 can provide forward push to the biopsy device at distal locations in the anatomy, for example, with a grasper or cup-like biopsy device that can be operated in a remote location or where tortuosity in the anatomy provides little pushability for the physician at the proximal location of the system.

Once everted the everting catheter system 2 can allow the operator to manually push, rotate, or actuate the biopsy mechanism for tissue collection. Once everted into the target tissue 90 region and tissue sample collection is completed, the everting balloon 12 can be inverted back over the tissue collection area 100 of the biopsy device, for example, protecting the tissue from contamination of adjacent tissues during device removal or device transport to the examination area.

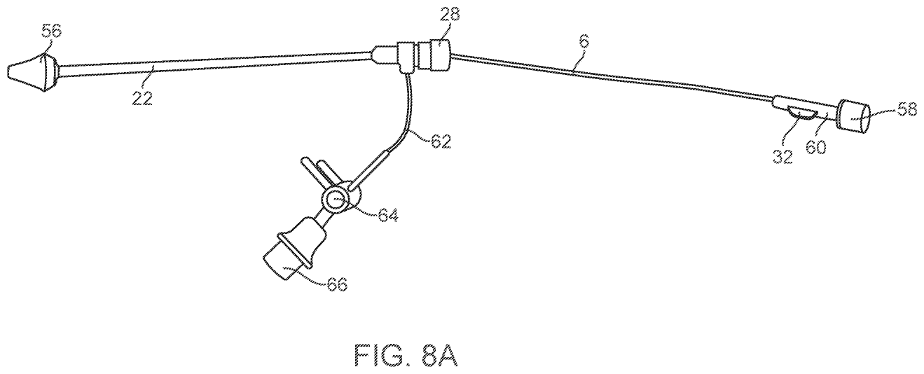

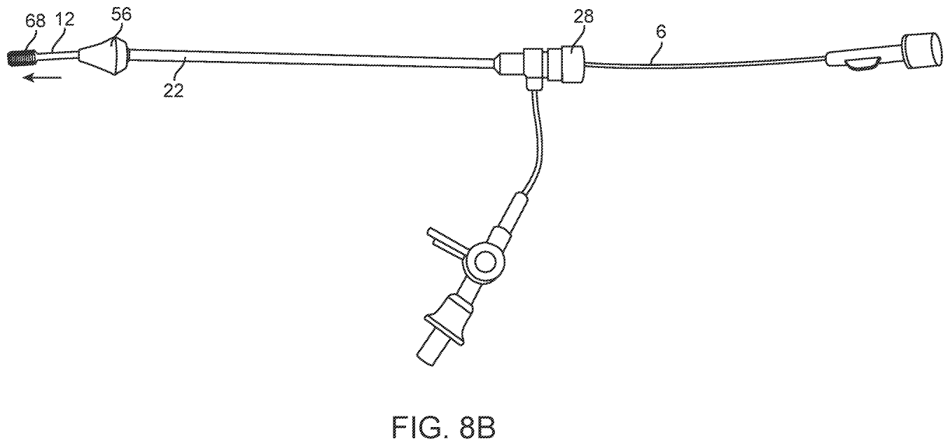

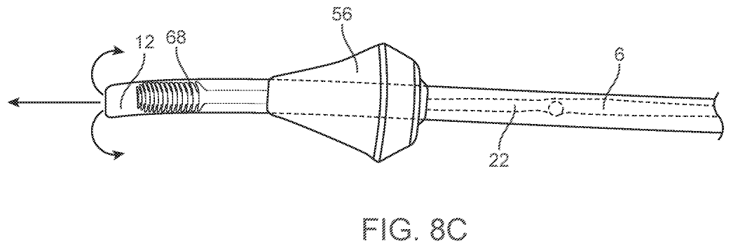

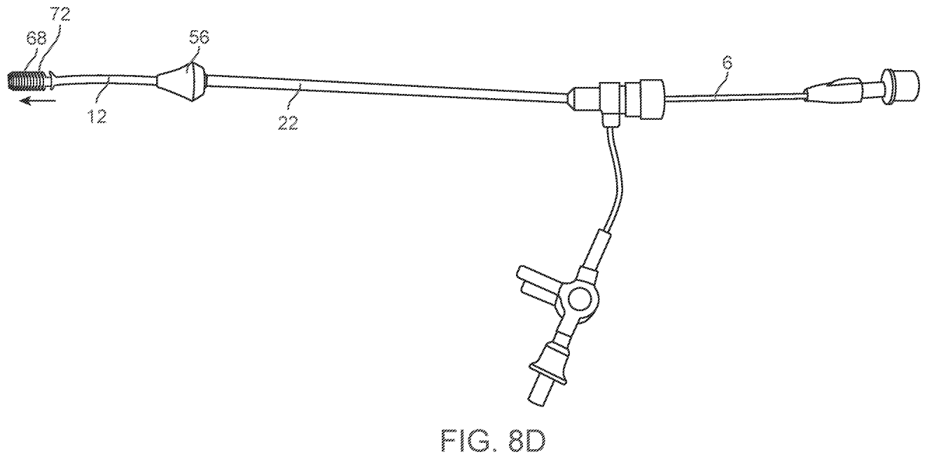

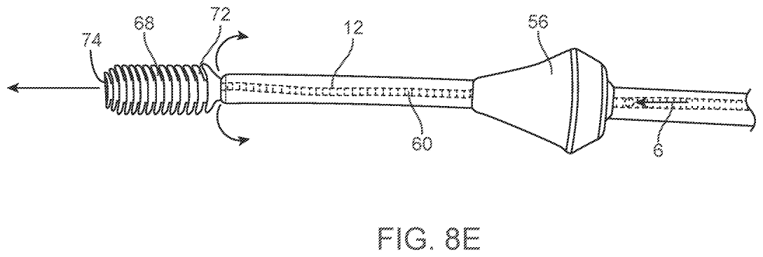

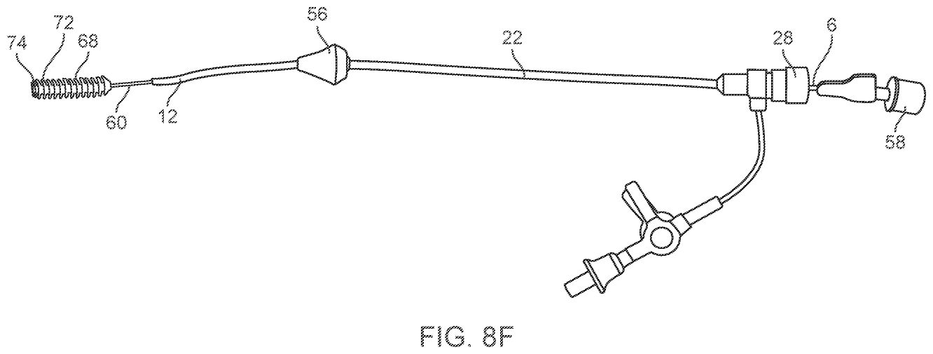

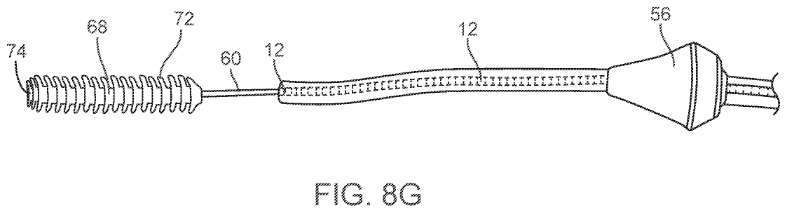

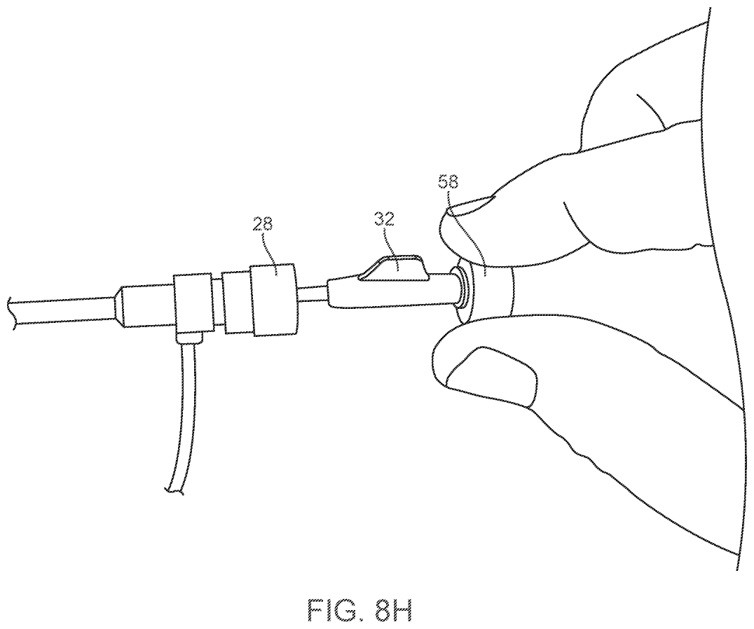

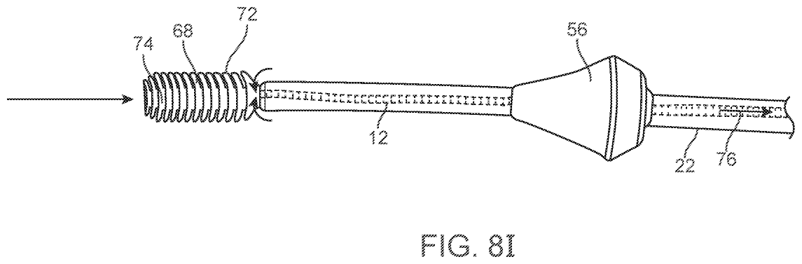

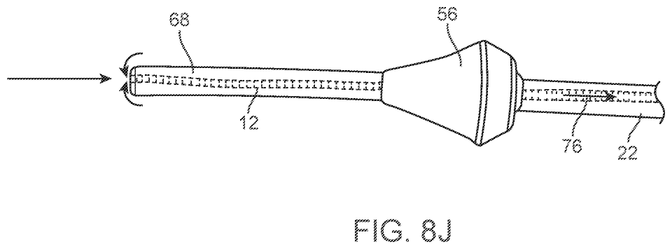

FIG. 8a illustrates that an everting catheter system 2 with an integrated cytology brush 68-type biopsy device can be used for cytology or tissue collection, for example, for endometrial biopsy procedures, biopsies of the GI tract, nasal cavity, bladder, ureter, urethra, esophagus, lungs, colon, other bodily cavities and lumens, or combinations thereof.

The everting catheter has an acorn tip 56 on the distal end of the outer catheter 22 which is designed to facilitate placement of the device at the exocervix of the patient. Within the outer catheter 22 is the inverted everting balloon 12 membrane with a cytology brush 68 housed within the interior of the inverted balloon. In practice the device can be provided to the clinical facility with the cytology brush 68 already housed within the outer catheter 22 or outside the everting system if the product is delivered with the everting catheter in the fully everted state. The proximal end 42 of the outer catheter 22 contains a valve fitting with o-ring seal. Connected to the valve fitting is an extension tube 62 with stopcock 64 and check valve 66 for providing pressurization media to the internal annulus of the outer catheter 22. The stopcock 64 can have a side port connection to the pressurization source. The pressurization media can be saline, saline warmed to body temperature, contrast media, echogenic-enhanced media with air bubbles, gas, air, or a combination of gas, air, and fluid. Exiting the valve fitting and o-ring on the proximal side is the inner catheter 6. The pressurization media can be supplied by syringe, inflation device, or other supply source of pressurized media. The inner catheter 6 extends into the outer catheter 22 and is connected to the everting balloon 12 on its distal end 46. The everting balloon 12 is connected to the distal end of the outer catheter 22 on its other end. On the proximal end 42 of the inner catheter 6 is a proximal hub 32. Within the inner catheter 6 and everting balloon 12 is a cytology brush 68 that extends from the proximal end 42 of the inner catheter 6 to beyond the distal end of the inner catheter 6. A knob 58 or handle can be on the proximal end 42 of the cytology brush 68 connected to the central wire of the cytology brush 60 for allowing manual manipulation of the cytology brush 68 when the everting catheter system 2 is in the everted state.

FIG. 8b illustrates the everting catheter with integrated biopsy brush that has been pressurized by a pressurization source (not shown) and with the inner catheter 6 slightly advanced or translated into the valve fitting resulting in the eversion of the everting balloon 12 out of the distal end of the outer catheter 22.

FIG. 8c illustrates a close up view of the everted balloon extending from the distal end of the outer catheter 22 and acorn tip 56. The everting balloon 12 is slightly extended and the cytology brush 68 is contained within the interior of everting balloon 12 as indicated. The everting balloon 12 delivers the biopsy brush to the target tissue 90 without exposure to unwanted or non-targeted tissue. This is determined by the length of everted balloon to the target tissue 90. As an example for endometrial biopsy, the overall everting balloon 12 length can be 3.5 cm from the distal end of the acorn tip 56 to the greatest extension of the everted balloon. This will ensure that the biopsy brush does not get exposed to the endocervical canal tissue, cervical mucus, or fluid, and instead will be only exposed to the intended uterine cavity 110. This is particularly useful in the case of endometrial biopsy in which cervical mucus is typically present and standard catheter systems can either pick up or scrape cervical mucus into the uterine cavity 110. The cervical mucus may confound the endometrial sampling results.

FIG. 8d illustrates an everting catheter with integrated biopsy brush slightly protruding from the everted balloon. The everting balloon 12 has advanced the biopsy brush and if in the anatomy, it is now exposed to the target tissue 90. The biopsy brush as a rounded distal tip and soft wire central body construction so that it can conform to the anatomy. The wire can be made from stainless steel, nitinol, nylon, delrin, or other polymer. The wire is typically a composite of two to three wires that are wound in a coil that envelopes the bristles 72 of the brush at the distal end 46. Alternatively the wire can be made from a single mandrel of metal or polymer in which the bristles 72 of the brush are bonded, wrapped, or molded into placed. Additionally, the wire or mandrel can be replaced with a tube with a central lumen 88 that can be used for the instillation or injection of fluid or media. The bristles 72 on the brush can be made from dacron, polyester, polypropylene, or other fabric or polymer. The biopsy brush in this example is shown with a knob 58 or handle at its proximal end 42. Alternatively the knob 58 can be configured with a port for the injection of fluid or media through a central lumen 88 within the biopsy brush. Further, the proximal hub 32 of the inner catheter 6 can contain a valve fitting and extension tube 62 for the instillation or injection of fluid or media through the interior lumen of the inner catheter 6. The instillation or injection of fluid or media can distend or rinse the bodily cavity, or the fluid can be used to wash the biopsy brush after specimen collection. Or additionally, the instillation or injection of fluid or media can be used to wash the everting balloon 12 membrane after specimen collection. And additionally, the washing of the everting balloon 12 membrane can be done as a separate step after tissue or cytology specimen collection of the brush.

FIG. 8e is a close up view of the biopsy brush protruding beyond the everting balloon 12. The bristles 72 of the brush become released from the enveloping everting balloon 12 membrane. Since the eversion of the everting balloon 12 is not yet complete, there can be bristles 72 visible within the everting balloon 12 membrane in a flattened or compressed state. The stainless steel wire composite of the biopsy brush central body is also visible within the everting balloon 12. The everting balloon 12 performs all of the manual advancement of the biopsy brush into the target tissue 90 region. In the example of endometrial biopsy, the biopsy brush is advanced further into the uterine cavity 110 and within the endometrium. At the very distal end of the biopsy brush, a round brush tip 74 provides a blunt surface to mitigate penetration into the wall or myometrium of the uterine cavity 110. The round tip can be made from HDPE (Teflon), Delrin, nylon, polyurethane, thermoplastic polyurethane, silicone, metal, other polymers, or combinations thereof. The round tip can also be lubriciously coated to facilitate movement of the brush within the anatomy. The round tip can have an outer diameter from about 0.5 mm to about 3.5 mm.

FIG. 8f shows the everting catheter with integrated biopsy brush after full eversion of the everting balloon 12. The biopsy brush is now fully extended from the everting balloon 12 and is no longer enveloped by the everting balloon 12. The brush can be manually manipulated by the physician either forward and back, or rotated, or both motions simultaneously. The movements or agitation of the brush within the target tissue 90 environment serves to sample more tissue for specimen collection or collect tissue in a wider area. For the example of endometrial biopsy, the physician will want to rotate and pump the brush forward and backward slightly to increase the amount of endometrium within or on the bristles 72 of the brush. The sampling procedure may be done under the visualization guidance of ultrasound or fluoroscopy. Ultrasound or fluoroscopic visualization can be enhanced with saline or contrast media distension which can be supplied by a valve fitting and extension tube 62 on the proximal hub 32 of the inner catheter 6 (not shown).

The ability to manually manipulate the distal end of the biopsy brush may not be necessary. The everting balloon 12 can be everted fully to expose the biopsy brush or device, followed by inversion of the everting balloon 12 to contain the tissue specimen for further diagnostic examination without the manual manipulation.

FIG. 8g is a close up of the biopsy brush extended beyond the distal end of the everting balloon 12. The bristles 72 of the brush are radially extended since they are no longer enveloped by the everting balloon 12. The pressurization state of the everting balloon 12 can be clearly seen in the photo. The everting balloon 12 can be fully everted and fully inflated. The distal end of the everting balloon 12 can be fully extended.

FIG. 8h is a close up view of the knob 58 of the cytology brush 68 and proximal end 42 of the inner catheter 6. The physician can manually manipulate the knob 58 of the cytology brush 68 to rotate or advance and retract the brush from the distal end of the everted balloon.

FIG. 8i shows a close up of the cytology brush 68 at the beginning of the inversion step. The bristles 72 of the brush can be seen being enveloped and flattened by the inverting balloon. As the balloon inverts and captures the bristles 72 of the brush, the cytology specimen are contained with the system. Inversion is performed by the user by retracting 76 the inner catheter 6 from the valve fitting and outer catheter 22. By inverting 92 the everting balloon 12, the biopsy brush is not exposed to non-target tissue. Alternatively, the entire system can be removed in one motion with or without pressurization (at negative pressure as an example) in the annulus of the outer catheter 22.

FIG. 8j shows a close up view of the cytology brush 68 fully enveloped during inversion of the everting balloon 12. The everting balloon 12 at this point in the inversion process has completely enveloped the bristles 72 of the biopsy brush. Further inversion of the everting balloon 12 and retraction of the inner catheter 6 will completely translate the everting balloon 12 into the outer catheter 22.

The everting catheter system 2 can have an integrated biopsy device deployed through the inner lumen 10. The biopsy device can have a curette 84 or shaver that can exit the distal end of the everting balloon 12. The biopsy device can have vacuum assist or have no vacuum assist. For example, the everting balloon 12 advancement can force the curette 84 to protrude into or on the target tissue 90. As described above for the biopsy brush, the everting balloon 12 can envelopes the curette 84 during the access step of the procedure. The everting balloon 12 during the inversion step can roll and assist in the capture of a tissue specimen.

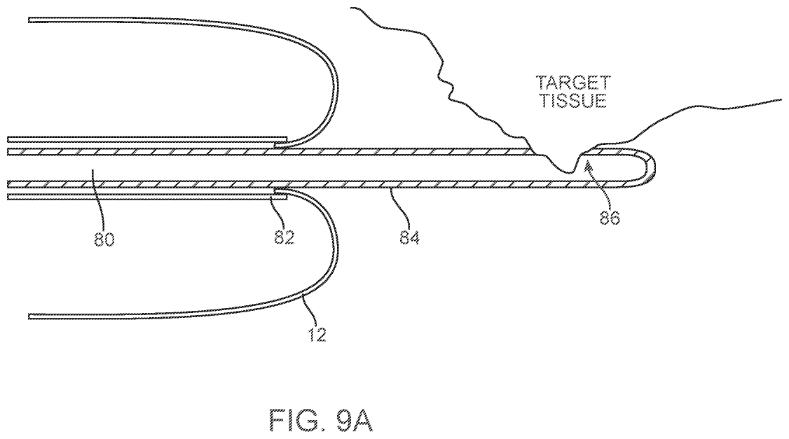

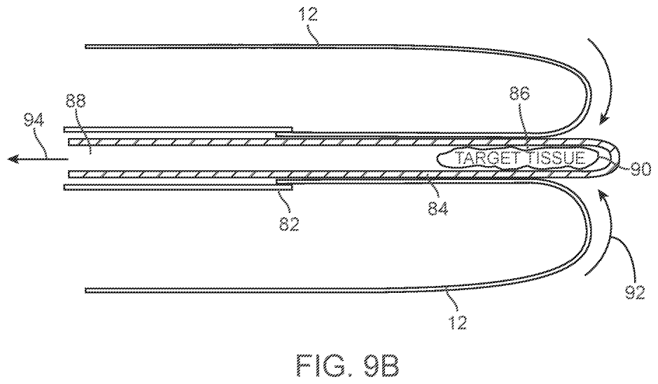

FIG. 9a illustrates that the distal end of an everting balloon 12 can have a curette 84 extending beyond the inner catheter distal end 82 and the everting balloon 12 distal end. The curette 84 can be translated as a result of the everting balloon 12 and inner catheter 6 pulling, and then pushing the curette 84 towards the target tissue 90. The curette 84 can have one or more side holes 86. Target tissue 90 for specimen collection may get scraped or trapped within the side hole 86, and/or be drawn into the side hole 86 by vacuum or aspiration pressure within the curette central lumen 80. The vacuum or aspiration source can be connected to the proximal end 42 (not shown) of the curette 84. The vacuum source can be replaced or substituted by an irrigation source for facilitating tissue removal at the examination site for diagnostic testing.

FIG. 9b illustrates the curette 84 and target tissue 90 during the inversion step of the procedure. The everting balloon 12 can facilitate the capture of target tissue 90 within the central lumen 88 and/or side hole 86 of the curette 84 as the everting balloon 12 inverts. Once the curette 84 is completely enveloped by the everting balloon 12 during the inversion process, the collected specimen of target tissue 90 can be isolated from other anatomical tissues and protected for transport to the examination area. At the examination area, the everting balloon 12 can be everted to expose the curette 84 and target tissue 90 for diagnostic examination. With the curette vacuum 94 removed, for example by detaching the curette vacuum 94 source, materials within the curette 84 can be flushed out of the curette 84 with irrigation media through the central lumen 88 of the curette 80.

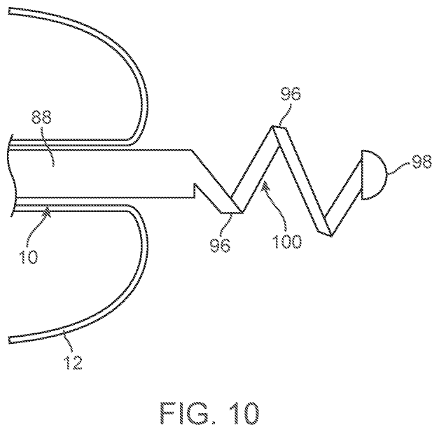

FIG. 10 illustrates that the integrated biopsy device within an everting catheter system 2 can be a shaver. The shaver can extend beyond the distal end of the everting balloon 12, for example, once the target tissue 90 is accessed. The shaver can radially and/or longitudinally open after being extended and released from the inner lumen of the everting balloon 12. The shaver can simultaneously get advanced by the everting balloon 12 during the everting 116 step. The shaver can may be manipulated (e.g., advanced, retracted, and rotated) by the user at the full eversion state. The shaver can have shaving projections that can close when the everting balloon 12 is inverted and the shaver projections 96 are pulled into the inner lumen 10, and can open after the shaver projections 96 exit the inner lumen 10. The shaver can have a target tissue 90 specimen contained within the collection area 100 of the shaver.

The shaver can have or be connected to a vacuum source on the proximal end 42 of the shaver to pull or hold tissue samples or otherwise facilitate tissue collection, and an irrigation source for clearing out collected tissue at the examination step for diagnostic testing.

The everting catheter system 2 can have an integrated biopsy device, for example a grasper (e.g., grasper jaws), loop, biopsy cup, or combinations thereof. The everting balloon 12 can provide access to the target tissue 90 within a bodily cavity or lumen. The everting balloon 12 can protect the surfaces of the biopsy device from unintended or premature tissue exposure. Once at the desired location (e.g., target site) within the anatomy, for example, as defined by the length of the everting balloon 12 and its advancement within the body, the everting balloon 12 can extend the biopsy device within the target tissue 90. As the biopsy device is extended, the grasper jaws can open, and/or the loop can enlarge to a larger circumference, and/or the biopsy cup can open. At a fully everted state, the biopsy device and be manipulated by the user at the proximal end 42 of the system. A vacuum source can be connected to the inner or central lumen 88 of the biopsy device to facilitate tissue collection (e.g., to suck tissue into the biopsy device). The inversion of the everting balloon 12 can squeeze closed the jaws of the grasper, shrink the circumference of the loop, or close the biopsy cup when pulled inside of the inner lumen of the everting balloon 12. Continued inversion of the everting balloon 12 can pull the biopsy device further into the inner lumen 10 and completely envelop the biopsy device, for example, covering it from potential contamination of unintended tissues. Complete inversion of the everting balloon 12 can protect the collected tissue specimen for transport to the examination area. Once the everting balloon 12 is re-everted to expose the biopsy device, an irrigation source can be connected to the proximal end 42 of the biopsy device to facilitate tissue removal for diagnostic testing at the examination area.

The everting catheter system 2 can have an aspiration-type biopsy device. The everting catheter system 2 can have an integrated aspiration source within the inner catheter 6. The inner catheter 6 can have a piston connected to a knob 58 of the aspiration device at the proximal end 42 of the inner catheter 6. Once the everting catheter has access to the bodily cavity or lumen and reached the target tissue 90, the knob 58 of the aspiration device at the proximal end 42 of the inner catheter 6 can be retracted. The translation of the knob 58 can pull vacuum pressure within the inner catheter 6 lumen drawing tissue, fluid, cellular matter, and combinations thereof, into the lumen.

Once fully retracted, the everting balloon 12 can be inverted to close off the distal end opening of the inner catheter 6. The closed distal end opening of the inner catheter 6 can contain the aspirated materials within the inner lumen of the inner catheter 6, protect the collected tissue specimen from contamination of unintended tissues or bodily fluids, provide a closed system for the collected tissue specimen until delivered to the examination area, evert the everting balloon 12 into the examination site to re-expose the distal end opening of the inner catheter 6 to allow the tissue specimen to be expelled from the inner catheter 6 by advancing the knob 58 of the aspiration device to act as a displacement piston to remove the collected tissue, or combinations thereof.

Any elements described herein as singular can be pluralized (i.e., anything described as “one” can be more than one). Any species element of a genus element can have the characteristics or elements of any other species element of that genus. “Dilation” and “dilatation” are used interchangeably herein. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

We claim:

1. A method for accessing a body cavity or lumen in a body comprising:
inserting an everting catheter system into the body, wherein the everting catheter system comprises an inner catheter defining an inner lumen, an outer catheter, and an everting balloon defining a balloon lumen, and wherein the inner catheter is in the balloon lumen;
deploying the everting balloon out of a distal end of the everting catheter system and into the body cavity or lumen, wherein deploying comprises positioning the inner catheter in the body cavity or lumen in a first profile that is smaller than a second profile, wherein the inner catheter has an expandable portion and a non-expandable portion positioned proximally from the expandable portion, wherein the inner catheter is pleated or folded longitudinally along the inner catheter and within the expandable portion when the inner catheter is in the first profile;
inserting an instrument through the inner lumen, and wherein the inner catheter has the second profile around the instrument when the instrument is inserted in the inner lumen; and tearing the everting balloon; and
rotating the instrument with respect to the everting balloon.

2. The method of claim 1, wherein the inner catheter is radially unexpanded in the first profile and radially expanded in the second profile.

3. The method of claim 1, wherein the instrument comprises an endoscope.

4. The method of claim 1, wherein the instrument comprises a cytology brush.

5. The method of claim 1, wherein the instrument comprises an IUD inserter.

6. The method of claim 1, wherein the instrument comprises a shaver.

7. The method of claim 1, wherein the instrument comprises an aspiration-type biopsy device.

8. The method of claim 1, wherein when the inner catheter is in the second profile, the everting balloon is torn at an intentional weakness in a wall of the everting balloon.

9. The method of claim 8, wherein the intentional weakness comprises a perforation.

10. The method of claim 8, wherein the tearing of the everting balloon comprises using a mechanical implement.

11. A method for accessing a body cavity or lumen in a body comprising:
inserting an everting catheter system into the body, wherein the everting catheter system comprises an inner catheter defining an inner lumen, an outer catheter, and an everting balloon defining a balloon lumen, and wherein the inner catheter is in the balloon lumen;
deploying the everting balloon out of a distal end of the everting catheter system and into the body cavity or lumen, wherein deploying comprises positioning the inner catheter in the body cavity or lumen in a first profile that is smaller than a second profile, wherein the inner catheter has an expandable portion and a non-expandable portion positioned proximally from the expandable portion, wherein the inner catheter is pleated or folded longitudinally along the inner catheter and within the expandable portion when the inner catheter is in the first profile;
inserting an IUD inserter through the inner lumen; and
rotating the IUD inserter with respect to the everting balloon.

12. The method of claim 11, wherein the inner catheter is radially unexpanded in the first profile and radially expanded in the second profile.

13. The method of claim 11, wherein when the inner catheter is in the second profile, the everting balloon is torn at an intentional weakness in a wall of the everting balloon.

14. The method of claim 12, wherein the intentional weakness comprises a perforation.

15. The method of claim 12, wherein the tearing of the everting balloon comprises using a mechanical implement.

16. A method for accessing a body cavity or lumen in a body comprising:
inserting an everting catheter system into the body, wherein the everting catheter system comprises an inner catheter defining an inner lumen, an outer catheter, and an everting balloon defining a balloon lumen, and wherein the inner catheter is in the balloon lumen;
deploying the everting balloon out of a distal end of the everting catheter system and into the body cavity or lumen, wherein deploying comprises positioning the inner catheter in the body cavity or lumen in a first profile that is smaller than a second profile, wherein the inner catheter has an expandable portion and a non-expandable portion positioned proximally from the expandable portion, wherein the inner catheter is pleated or folded longitudinally along the inner catheter and within the expandable portion when the inner catheter is in the first profile;
inserting an instrument through the inner lumen, wherein the inner catheter is radially unexpanded in the first profile and radially expanded in the second profile; and
rotating the instrument with respect to the everting balloon.

17. The method of claim 16, wherein the instrument comprises an endoscope.

18. The method of claim 16, wherein the instrument comprises a cytology brush.

19. The method of claim 16, wherein the instrument comprises an IUD inserter.

20. The method of claim 16, wherein when the inner catheter is in the second profile, the everting balloon is torn at an intentional weakness in a wall of the everting balloon.

21. The method of claim 20, wherein the intentional weakness comprises a perforation.
